(19) United States
(12) Patent Application Publication
(10) Pub. No.: US 2020/0184644 A1
(43) Pub. Date: Jun. 11, 2020

(54) MEDICAL IMAGE PROCESSING DEVICE AND MEDICAL IMAGE PROCESSING METHOD

(71) Applicant: FUJIFILM Corporation, Tokyo (JP)

(72) Inventor: Chihiro UEDA, Kanagawa (JP)

(73) Assignee: FUJIFILM Corporation, Tokyo (JP)

(21) Appl. No.: 16/790,659

(22) Filed: Feb. 13, 2020

Related U.S. Application Data
(63) Continuation of application No. PCT/JP2018/029384, filed on Aug. 6, 2018.

(30) Foreign Application Priority Data
Aug. 24, 2017 (JP) 2017-160986

(51) Int. Cl. G06T 7/00; A61B 1/045

(52) U.S. Cl. CPC G06T 7/0012; G06T 2207/30004; G06T 2207/10021; A61B 1/045

(57) ABSTRACT

Provided are a medical image processing device and a medical image processing method which can accurately grasp a reproduction status of a video of a medical image by a user such as a doctor. A video reproduction control unit that controls reproduction of a video of a medical image in a display device; a reproduction number control unit that counts the reproduction number of the video by the video reproduction control unit, and counts up the reproduction number of the video in a case where the video is reproduced until a specific frame image among a plurality of frame images constituting the video; and an information display control unit that causes the display device to display the reproduction number of the video, which is counted by the reproduction number control unit are included.

### CROSS-REFERENCE TO RELATED APPLICATIONS

This application is a Continuation of PCT International Application No. PCT/JP2018/029384 filed on Aug. 6, 2018, which claims priority under 35 U.S.C § 119(a) to Japanese Patent Application No. 2017-160986 filed on Aug. 24, 2017. Each of the above application(s) is hereby expressly incorporated by reference, in its entirety, into the present application.

### BACKGROUND OF THE INVENTION

### 1. Field of the Invention

The invention relates to a medical image processing device and a medical image processing method which reproduce a video of medical images.

### 2. Description of the Related Art

In the related art, endoscopic examination using an electronic endoscope (hereinafter, simply abbreviated to an endoscope) has been performed in the medical field. In the endoscopic examination, observation of an observation site is performed on the basis of a reflected light image which is acquired by applying illumination light to the observation site in an object to be examined and performing image pick-up of the reflected light of the illumination light reflected from the observation site, for example. In this case, a video of the observation site is captured by the endoscope and the video of the observation site is obtained. This video is used as a more accurate diagnosis by a doctor, determination on presence or absence of a lesion or the like by an image analysis (video analysis), a medical report material, and the like.

Since the endoscopic examination is performed frequently, and accordingly the number of videos obtained in the endoscopic examination is increased, it is preferable that a doctor can check a reproduction status of each video at a glance. For example, JP2010-283477A and JP2000-350165A disclose that a reproduction number of each video is preserved as a history. In addition, JP2016-119590A discloses that in a case where a full video is reproduced, the reproduction number of the video is counted up.

### SUMMARY OF THE INVENTION

However, in the video of the medical image obtained in the endoscopic examination, a frame image including a region of interest such as a lesion is particularly important in the diagnosis or the like. Therefore, it can be said that in a case where the doctor has checked the frame image including the region of interest without reproducing the full video, the content of the video is substantially checked. Accordingly, as disclosed in JP2016-119590A, in a case where the reproduction number is counted up on condition of reproducing a full video, since the reproduction is not reflected to the reproduction number of a video even if the doctor has substantially checked the content of the video, it is difficult to accurately grasp the reproduction status of each video by the doctor.

The invention has been made in view of such circumstances, and an object of the invention is to provide a medical image processing device and a medical image processing method which can accurately grasp a reproduction status of a video of a medical image by a user such as a doctor.

A medical image processing device for achieving the object of the invention comprises a video reproduction control unit that controls reproduction of a video of a medical image in a display device; a reproduction number control unit that counts the reproduction number of the video by the video reproduction control unit, and counts up the reproduction number of the video in a case where the video is reproduced until a specific frame image among a plurality of frame images constituting the video; and an information display control unit that causes the display device to display the reproduction number of the video, which is counted by the reproduction number control unit.

With the medical image processing device, in a case where checking the content of the video by the user is substantially completed even if the full video is not reproduced, it is possible to count up the reproduction number of the video.

The medical image processing device according to another aspect of the invention further comprises a medical image analysis processing unit that analyzes whether a region of interest as a region to be noticed is included for each of the frame images, and determines the frame image including the region of interest as the specific frame image. In this manner, it is possible to determine a frame image which is highly necessary for an accurate diagnosis and checking by the user (doctor), as the specific frame image.

In the medical image processing device according to another aspect of the invention, the medical image analysis processing unit analyzes whether the region of interest is included in the frame image by an image analysis process using deep learning. In this manner, it is possible to determine a frame image which is highly necessary for a diagnosis and checking by the user (doctor), as the specific frame image.

In the medical image processing device according to another aspect of the invention, the video is acquired by a medical device having a function of performing capturing of the video and capturing of a static image during the capturing of the video, and the medical image processing device further comprises a timing information storage unit that stores timing information indicating a timing at which the capturing of the static image is executed during the capturing of the video in a case where the capturing of the static image is executed by the medical device, and an image specifying unit that determines the frame image captured at the timing at which the capturing of the static image is executed, as the specific frame image on the basis of the timing information stored in the timing information storage unit. In this manner, it is possible to determine a frame image which is highly necessary for a diagnosis and checking by the user (doctor), as the specific frame image.

The medical image processing device according to another aspect of the invention further comprises a reproduction number storage unit that stores the reproduction number of the video, which is counted by the reproduction number control unit. By storing the reproduction number of the video, checking and display of the reproduction number of the video can be performed at any time.

The medical image processing device according to another aspect of the invention further comprises a user specifying unit that specifies a user who executes reproduction of the video in the video reproduction control unit, the reproduction number control unit individually counts the reproduction number of the video for each user, and the reproduction number storage unit individually stores the reproduction number of the video for each user. In this manner, it is possible to easily accurately grasp the reproduction status of the video for each user.

In the medical image processing device according to another aspect of the invention, the information display control unit causes the display device to display reproduction presence/absence information indicating presence or absence of reproduction of the video. In this manner, it is possible to easily discriminate the presence/absence of the reproduction of the video by the user.

In the medical image processing device according to another aspect of the invention, the information display control unit causes the display device to display at least one of a numerical value or a graph indicating the reproduction number of the video, as the reproduction number of the video. In this manner, it is possible to easily grasp the reproduction number of the video.

In the medical image processing device according to another aspect of the invention, in a case where a plurality of videos are present, the information display control unit causes the display device to display the reproduction number of the video, for each of the videos. In this manner, it is possible to easily grasp the reproduction status of each video.

A medical image processing method for achieving the object of the invention comprises a video reproduction control step of controlling reproduction of a video of a medical image in a display device; a reproduction number control step of counting the reproduction number of the video reproduced in the video reproduction control step, and counting up the reproduction number of the video in a case where the video is reproduced until a specific frame image among a plurality of frame images constituting the video; and a display control step of causing the display device to display the reproduction number of the video, which is counted in the reproduction number control step.

A medical image processing device and a medical image processing method according to an embodiment of the invention can accurately grasp a reproduction status of a video of a medical image by a user such as a doctor.

### BRIEF DESCRIPTION OF THE DRAWINGS

FIG. 1 is a schematic diagram showing the entire configuration of an endoscope system.

FIG. 2 is a functional block diagram showing functions of a medical image processing device.

FIG. 3 is a schematic diagram of an image storage unit.

FIG. 4 is an explanatory diagram for describing an example of a specific frame image.

FIG. 5 is an explanatory diagram showing an example of a reproduction image selection screen.

FIG. 6 is an explanatory diagram for describing a reproduction control of a video by a reproduction control unit.

FIG. 7 is a schematic diagram of a reproduction number storage unit in the storage unit.

FIG. 8 is an explanatory diagram for describing counting-up of a reproduction number of a video by a reproduction number control unit in a case where a specific frame image is specified by a medical image analysis processing unit.

FIG. 9 is an explanatory diagram for describing counting-up of a reproduction number of a video by the reproduction number control unit in a case where a specific frame image is specified by an image specifying unit.

FIG. 10 is an explanatory diagram for describing updating of a reproduction number display by an information display control unit.

FIG. 11 is a flowchart showing a flow of reproducing a video and counting the reproduction number of the video in the endoscope system.

### DESCRIPTION OF THE PREFERRED EMBODIMENTS

[Entire Configuration of Endoscope System]

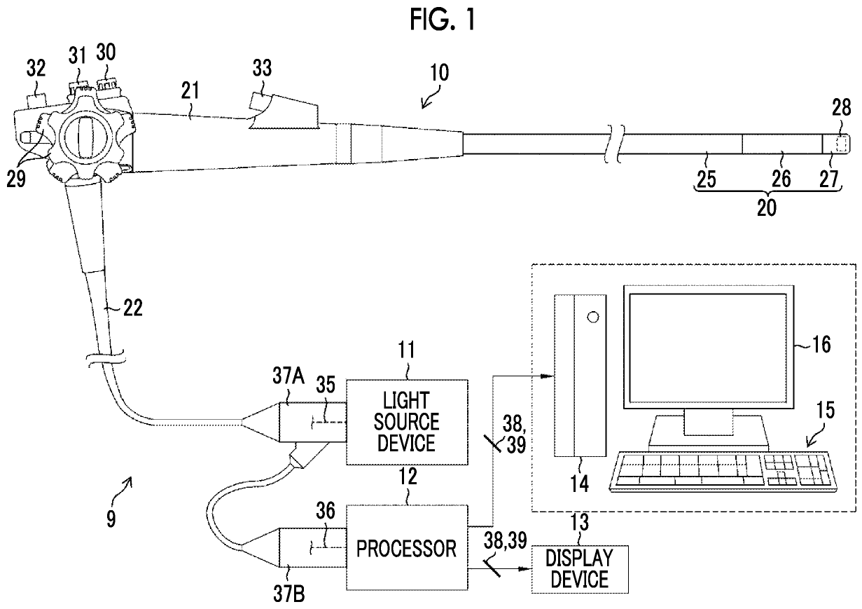

FIG. 1 is a schematic diagram showing the entire configuration of an endoscope system 9. As shown in FIG. 1, the endoscope system 9 includes an endoscope 10 which is an electronic endoscope, a light source device 11, a processor 12, a display device 13, a medical image processing device 14, an operation unit 15, and a display device 16.

The endoscope 10 corresponds to a medical device of an embodiment of the invention, and is a flexible endoscope, for example. The endoscope 10 includes an insertion part 20 that is to be inserted into an object to be examined and has a distal end and a proximal end, an operation part 21 that is provided to be continuous to the proximal end side of the insertion part 20 and is held by an operator to perform various operations, and a universal cord 22 that is provided to be continuous to the operation part 21.

The insertion part 20 is formed in a long shape with a small diameter as a whole. The insertion part 20 is configured by a soft portion 25 having flexibility, a bendable portion 26 that is bendable by an operation of the operation part 21, and a distal end portion 27 in which an imaging optical system (not shown), an image pick-up element 28, and the like are built, which are continuously provided in order from the proximal end side to the distal end side.

The image pick-up element 28 is a complementary metal oxide semiconductor (CMOS) type or charge coupled device (CCD) image pick-up element. Image light of an observation site is incident on an image pick-up surface of the image pick-up element 28 via an observation window (not shown) open in the distal end surface of the distal end portion 27 and the imaging optical system (not shown) disposed behind the observation window. The image pick-up element 28 performs image pick-up (conversion into electrical signals) of the image light of the observation site, which is incident on the image pick-up surface, and outputs an image pick-up signal.

Various operation members to be operated by an operator are provided in the operation part 21. Specifically, the operation part 21 is provided with two kinds of bendable operation knobs 29 used for the bendable operation of the bendable portion 26, an air/water supply button 30 for an air/water supply operation, and a suction button 31 for a suction operation. The operation part 21 is provided with a static image capturing instruction portion 32 for performing an imaging instruction of a static image 39 of the observation site, and a treatment tool inlet 33 through which a treatment tool (not shown) is inserted into a treatment tool passage (not shown) inserted into the insertion part 20.

The universal cord 22 is a connection cord for connecting the endoscope 10 to the light source device 11. The universal cord 22 includes a light guide 35, a signal cable 36, and a fluid tube (not shown) which are inserted into the insertion part 20. In addition, the end portion of the universal cord 22 is provided with a connector 37A connected to the light source device 11, and a connector 37B which branches from the connector 37A and is connected to the processor 12.

The connector 37A is connected to the light source device 11 so that the light guide 35 and the fluid tube (not shown) are inserted into the light source device 11. In this manner, required illumination light, water, and air are supplied from the light source device 11 to the endoscope 10 via the light guide 35 and the fluid tube (not shown). As a result, illumination light is emitted to the observation site from the illumination window (not shown) of the distal end surface of the distal end portion 27. According to a pressing operation of the air/water supply button 30, air or water is sprayed from an air/water supply nozzle (not shown) of the distal end surface of the distal end portion 27 toward the observation window (not shown) of the distal end surface.

The connector 37B is connected to the processor 12 so that the signal cable 36 and the processor 12 are electrically connected to each other. In this manner, an image pick-up signal of the observation site is output from the image pick-up element 28 of the endoscope 10 to the processor 12 via the signal cable 36, and a control signal is output from the processor 12 to the endoscope 10 via the signal cable 36.

In this embodiment, a flexible endoscope is described as an example of the endoscope 10, but various endoscopes which can capture a video of the observation site, such as a rigid endoscope may be used.

The light source device 11 supplies illumination light to the light guide 35 of the endoscope 10 via the connector 37A. As the illumination light, light in various wavelength ranges is selected according to the observation purpose, such as white light (light in white-light wavelength range or light in a plurality of wavelength ranges) or light in one or a plurality of specific wavelength ranges, or a combination thereof. The specific wavelength range is a range narrower than the white-light wavelength range.

A first example of the specific wavelength range is a blue-light wavelength range or a green-light wavelength range of a visible-light wavelength range, for example. The wavelength range of the first example includes a wavelength range of 390 nm to 450 nm or 530 nm to 550 nm, and light of the first example has a peak wavelength in a wavelength range of 390 nm to 450 nm or 530 nm to 550 nm.

A second example of the specific wavelength range is a red-light wavelength range of a visible-light wavelength range, for example. The wavelength range of the second example includes a wavelength range of 585 nm to 615 nm or 610 nm to 730 nm, and light of the second example has a peak wavelength in a wavelength range of 585 nm to 615 nm or 610 nm to 730 nm.

A third example of the specific wavelength range includes a wavelength range where a light absorption coefficient in oxyhemoglobin is different from that in reduced hemoglobin, and light of the third example has a peak wavelength in a wavelength range where a light absorption coefficient in oxyhemoglobin is different from that in reduced hemoglobin. The specific wavelength range of the third example includes a wavelength range of 400±10 nm, 440±10 nm, 470±10 nm, or 600 nm to 750 nm, and light of the third example has a peak wavelength in a wavelength range of 400±10 nm, 440±10 nm, 470±10 nm, or 600 nm to 750 nm.

A fourth example of the specific wavelength range is a wavelength range (390 nm to 470 nm) of excitation light which is used for observation (fluorescence observation) of fluorescence emitted by fluorescent materials in a living body and excites the fluorescent materials.

A fifth example of the specific wavelength range is an infrared wavelength range. The wavelength range of the fifth example includes a wavelength range of 790 nm to 820 nm or 905 nm to 970 nm, and light of the fifth example has a peak wavelength in a wavelength range of 790 nm to 820 nm or 905 nm to 970 nm.

The processor 12 controls the operation of the endoscope 10 via the connector 37B and the signal cable 36. In addition, the processor 12 generates a video 38 of the observation site on the basis of an image pick-up signal acquired from the image pick-up element 28 of the endoscope 10 via the connector 37B and the signal cable 36. Further, in a case where the static image capturing instruction portion 32 is operated in the operation part 21 of the endoscope 10, the processor 12 generates a static image 39 of the observation site on the basis of the image pick-up signal acquired from the image pick-up element 28 in parallel with the generation of the video 38. The static image 39 may be generated to have higher resolution than the video 38.

The video 38 and the static image 39 are in-vivo images captured in the object to be examined, that is, in the living body. Further, in a case where the video 38 and the static image 39 are images acquired by light (special light) in the above-described specific wavelength range, the video 38 and the static image 39 are special light images. The processor 12 outputs the generated video 38 and static image 39 to each of the display device 13 and the medical image processing device 14.

The processor 12 may generate (acquire) a special light image having information of the above-described specific wavelength range on the basis of a normal light image acquired by the above-described white light. In this case, the processor 12 functions as a special light image acquisition unit. The processor 12 acquires a signal of the specific wavelength range by performing an arithmetic operation based on color information about red, green, blue (RGB) or cyan, magenta, and yellow (CMY) included in the normal light image.

The processor 12 may generate a feature quantity image such as a known oxygen saturation image on the basis of at least one of the normal light image obtained by the above-described white light or the special light image obtained by the light (special light) in the above-described specific wavelength range. In this case, the processor 12 functions as a feature-quantity-image generation unit.

The display device 13 is connected to the processor 12 and displays the video 38 and the static image 39 input from the processor 12. The user (doctor) performs an advance/retraction operation of the insertion part 20 while checking the video 38 displayed on the display device 13, and in a case where a lesion or the like is found in the observation site, the user (doctor) operates the static image capturing instruction portion 32 to execute static image capturing of the observation site.

As the medical image processing device 14, for example, a personal computer is used in this embodiment. A keyboard, a mouse, or the like connected to the personal computer in a wired or wireless manner is used as the operation unit 15, and various monitors that can be connected to the personal computer are used as the display device 16.

In addition, a diagnosis support apparatus such as a work station (server) may be used as the medical image processing device 14, and in this case, the operation unit 15 and the display device 16 are provided to each of a plurality of terminals connected to the work station. Further, as the medical image processing device 14, a medical service support device that supports the creation of a medical report or the like may be used, for example.

The medical image processing device 14 performs acquisition and storage of the video 38 and the static image 39, reproduction control of the video 38 and the static image 39, counting the reproduction number of the video 38, and displaying the counting result. The operation unit 15 is used for inputting an operation instruction to the medical image processing device 14. The display device 16 displays the video 38 and the static image 39 or displays the reproduction number of the video 38 or the like under the control of the medical image processing device 14.

[Function of Medical Image Processing Device]

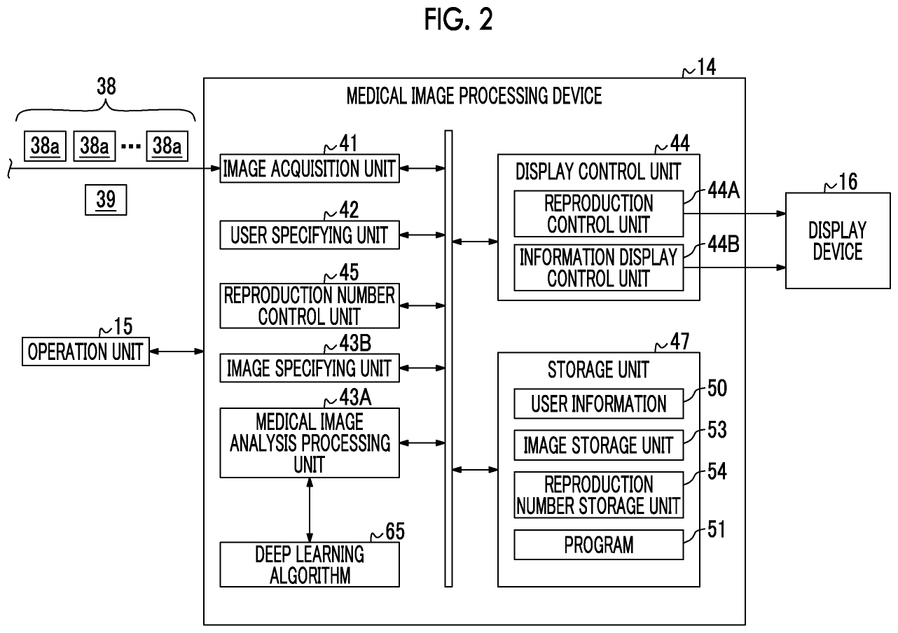

FIG. 2 is a functional block diagram showing functions of the medical image processing device 14. As shown in FIG. 2, a hardware structure of the control unit which executes various controls of the medical image processing device 14 including the reproduction control of the video 38, the counting of the reproduction number of the video 38, and the display of the counting result of the reproduction number is various processors described below. The various processors include a central processing unit (CPU) as a general-purpose processor executing software (program) and functioning as various processing units, a programmable logic device (PLD) as a processor of which the circuit configuration can be changed after manufacturing such as a field programmable gate array (FPGA), a dedicated electrical circuit as a processor having a circuit configuration designed exclusively for executing specific processing such as an application specific integrated circuit (ASIC).

One processing unit may be configured to include one processor among these various processors, or may be configured to include two or more same or different kinds of processors (for example, a combination of a plurality of FPGAs or a combination of the CPU and the FPGA). Further, a plurality of control units may be configured by one processor. As an example where a plurality of control units are configured by one processor, first, there is an aspect where one processor is configured by a combination of one or more CPUs and software as typified by a computer, such as a client or a server, and this processor functions as a plurality of control units. Second, there is an aspect where a processor fulfilling the functions of the entire system including a plurality of control units by one integrated circuit (IC) chip as typified by a system on chip (SoC) or the like is used. In this way, various control units are configured using one or more of the above-described various processors as hardware structures.

The processor (not shown) of the medical image processing device 14 functions as an image acquisition unit 41, a user specifying unit 42, a medical image analysis processing unit 43A, an image specifying unit 43B, a display control unit 44, and a reproduction number control unit 45 on the basis of the program 51. In addition, the medical image processing device 14 is provided with a storage unit 47 that stores information relating to various controls of the medical image processing device 14.

The storage unit 47 stores user information 50 and a program 51, and is provided with an image storage unit 53 and a reproduction number storage unit 54. The user information 50 is information in which a user identification (ID) unique to a user such as a doctor who uses the medical image processing device 14 and a password determined for each user ID are associated. The program 51 is an application program for causing the medical image processing device 14 to execute the reproduction control of the video 38, the counting of the reproduction number of the video 38, the display of the counting result, and the like.

As will be described in detail below, the image storage unit 53 stores the video 38 and the static image 39 acquired by the medical image processing device 14. Further, as will be described in detail below, the reproduction number storage unit 54 stores the reproduction number of the video 38 for each user.

In this embodiment, the storage unit 47 is provided in the medical image processing device 14, but the storage unit 47 may be provided in a server or a database on the Internet. Therefore, the medical image processing device of the embodiment of the invention also includes a medical image processing system configured by a plurality of devices.

The image acquisition unit 41 acquires the video 38 from the processor 12 using an image input/output interface (not shown) connected to the processor 12 in a wired or wireless manner. In addition, in a case where the static image 39 is captured while the video 38 is being captured in the endoscope 10, the image acquisition unit 41 acquires the video 38 and the static image 39 from the processor 12. Then, the image acquisition unit 41 stores the acquired video 38 and static image 39 in the image storage unit 53 in the storage unit 47. The reference signs 38a in the drawing indicate a plurality of frame images constituting the video 38.

The image acquisition unit 41 may acquire the video 38 and the static image 39 via various information storage mediums such as a memory card instead of directly acquiring the video 38 and the static image 39 from the processor 12. Further, the image acquisition unit 41 may acquire, via the Internet, the video 38 and the static image 39 uploaded to a server or a database on the Internet.

In a case where the image acquisition unit 41 acquires the special light image having information of the specific wavelength range, as the video 38 and the static image 39, the image acquisition unit 41 functions as the special light image acquisition unit.

Further, in a case where static image capturing of the observation site is performed according to the operation of the static image capturing instruction portion 32, the image acquisition unit 41 may cause the image storage unit 53 to store the video 38 for one minute before and after the static image capturing (for one minute before the static image capturing to one minute after the static image capturing) without causing the image storage unit 53 to necessarily store the full video 38 input from the processor 12 or the like.

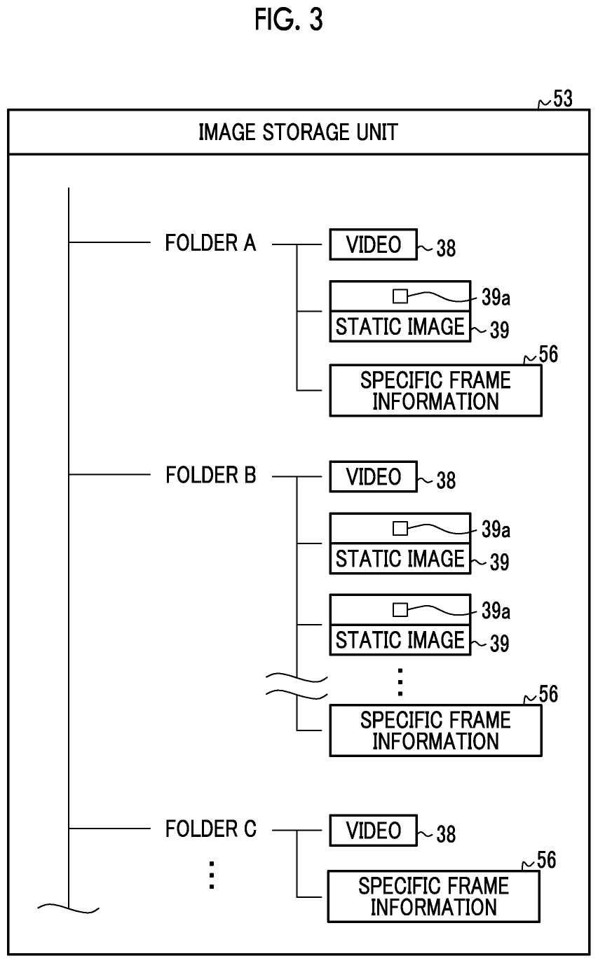

FIG. 3 is a schematic diagram of the image storage unit 53. As shown in FIG. 3, the image acquisition unit 41 stores the video 38 acquired from the processor 12 or the like separately for each folder (folder A, folder B, folder C, and the like) in the image storage unit 53. Further, in a case where the static image 39 is captured while the video 38 is being captured in the endoscope 10, the image acquisition unit 41 stores the static image 39 acquired together with the video 38 from the processor 12 in the same folder as the video 38.

The video 38 is shown as one file in the drawing, but in a case where the imaging time of the video 38 is long, the video 38 may be divided into a plurality of files.

In header information 39a of the static image 39, time information indicating an imaging timing at which the static image 39 is captured while the video 38 is being captured is recorded by the processor 12. Therefore, it is possible to discriminate the imaging timing by referring to the header information 39a. Accordingly, the header information 39a corresponds to timing information of an embodiment of the invention, and the image storage unit 53 functions as a timing information storage unit of an embodiment of the invention.

In this embodiment, the header information 39a of the static image 39 is used as the timing information of an embodiment of the invention, but the information may be stored in, for example, a header of the video 38 without being limited to the header information 39a as long as the imaging timing can be discriminated, and may be stored differently from the video 38 and the static image 39.

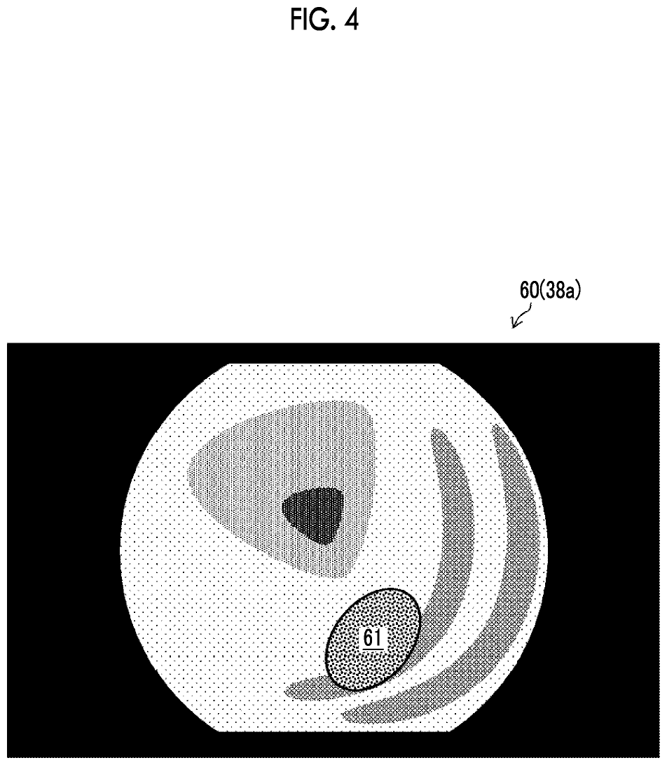

In each folder in the image storage unit 53, specific frame information 56 as the analysis result of the medical image analysis processing unit 43A and the image specifying unit 43B to be described below is stored. As will be described in detail below, the specific frame information 56 is information indicating a specific frame image 60 (refer to FIG. 4) included in a plurality of frame images 38a constituting the video 38 stored in the same folder.

Returning to FIG. 2, the user specifying unit 42 specifies a user when the user starts to use the medical image processing device 14. For example, the user specifying unit 42 controls the display control unit 44, which will be described below, to cause the display device 16 to display a login screen, and prompts the user to input the user ID and the password. Then, in a case where the user inputs the user ID and the password on the login screen, the user specifying unit 42 collates the input user ID and password with the user information 50 in the storage unit 47 to specify the user using the medical image processing device 14. Then, the user specifying unit 42 outputs the specifying result of the user to each of the display control unit 44 and the reproduction number control unit 45.

Instead of specifying a user on the basis of the user ID and the password, an ID card that the user owns may be used, or a known biometric authentication technique may be used.

The medical image analysis processing unit 43A and the image specifying unit 43B specify a predetermined specific frame image 60 (refer to FIG. 4) from among the plurality of frame images 38a constituting the video 38. The specific frame image 60 is an image that is highly necessary to be checked for an accurate diagnosis by the user (doctor).

The medical image analysis processing unit 43A analyzes whether a region of interest 61 (refer to FIG. 4) which is a region to be noticed is included for each frame image 38a, and determines the frame image 38a including the region of interest 61 as the specific frame image 60 (refer to FIG. 4).

FIG. 4 is an explanatory diagram for describing an example of the specific frame image 60. As shown in FIG. 4, the region of interest 61 is a lesion region occurring in the observation site, for example. In addition, in the region of interest 61, scars (treatment scar and inspection scar) left on the body wall of the observation site due to a treatment or inspection by a treatment tool or the like in the past are included in addition to the lesion region. Further, as the region of interest 61, a region where a treatment or inspection by a treatment tool or the like is performed, that is, a region where a target to be noticed such as a treatment tool or the like is shown is also included.

Returning to FIG. 2, whenever a new video 38 is stored in the image storage unit 53, the medical image analysis processing unit 43A of this embodiment analyzes whether the region of interest 61 is included for each frame image 38a by performing an image analysis process using deep learning on the basis of a deep learning algorithm 65. The deep learning algorithm 65 is an algorithm for recognizing whether the region of interest 61 is included in the frame image 38a through a known convolutional neural network method, that is, through repetition of a convolutional layer and a pooling layer, a fully connected layer, and an output layer. Since the image analysis process using deep learning is a known technique, specific description is omitted.

The medical image analysis processing unit 43A determines the frame image 38a including the region of interest 61 as the specific frame image 60, and stores information which can identify the specific frame image 60 in the video 38 (frame number or the like), as the specific frame information 56 in the same folder as the video 38.

In a case where the static image 39 is captured while the video 38 is being captured in the endoscope 10, the image specifying unit 43B determines the frame image 38a of the video 38 captured at an imaging timing at which the static image 39 is captured, as the specific frame image 60. Here, the static image 39 is captured often in a case where a lesion region has occurred in the observation site, in a case where a treatment scar or an inspection scar is present in the observation site, and in a case where a treatment or inspection by a treatment tool or the like is being performed on the observation site. Therefore, it is high likely for the region of interest 61 to be included in the frame image 38a of the video 38 captured at the imaging timing.

Whenever a new video 38 is stored in the image storage unit 53, the image specifying unit 43B checks whether the static image 39 is stored in the same folder as the video 38. Next, in a case where the static image 39 is stored in the same folder as the video 38, the image specifying unit 43B determines, as the specific frame image 60, the frame image 38a captured at the imaging timing, among the frame images 38a of the video 38 on the basis of the header information 39a of the static image 39. Then, similar to the medical image analysis processing unit 43A, the image specifying unit 43B stores information which can identify the specific frame image 60 in the video 38, as the specific frame information 56 in the same folder as the video 38.

In this manner, in the specific frame information 56 of this embodiment, both the information indicating the specific frame image 60 specified by the medical image analysis processing unit 43A and the information indicating the specific frame image 60 specified by the image specifying unit 43B are included.

The medical image processing device 14 of this embodiment includes both the medical image analysis processing unit 43A and the image specifying unit 43B, but may include only one of the medical image analysis processing unit 43A and the image specifying unit 43B.

The display control unit 44 controls an image display by the display device 16. The display control unit 44 functions as a reproduction control unit 44A and an information display control unit 44B.

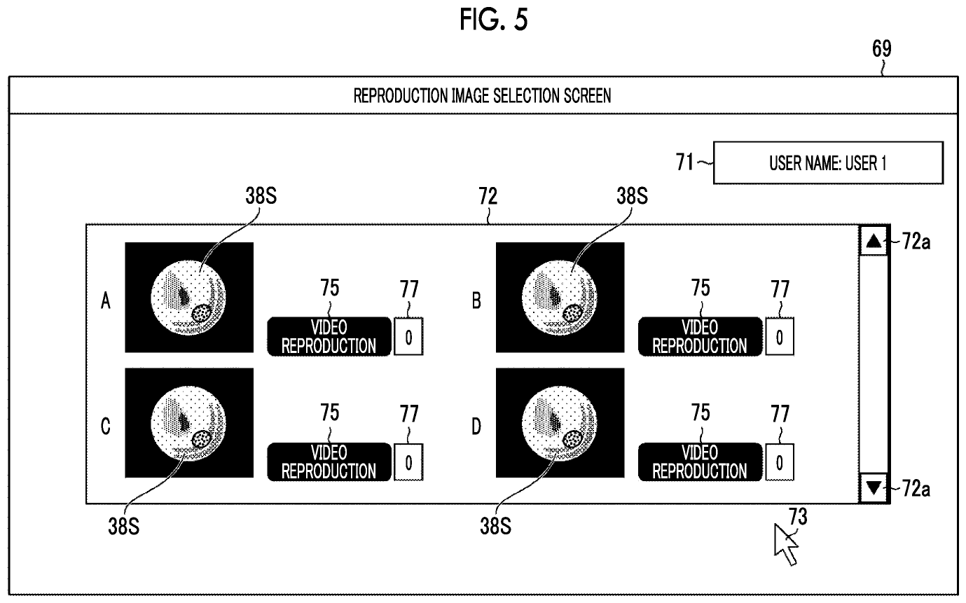

The reproduction control unit 44A corresponds to a video reproduction control unit of an embodiment of the invention, and performs a reproduction control of causing the display device 16 to reproduce the video 38 (static image 39 is also possible) stored in each folder in the image storage unit 53. In a case where a reproduction image selection screen display operation is performed in the operation unit 15, the reproduction control unit 44A causes the display device 16 to display a reproduction image selection screen 69 (refer to FIG. 5) on the basis of the program 51 and the image storage unit 53 in the storage unit 47.

FIG. 5 is an explanatory diagram showing an example of the reproduction image selection screen 69. In FIG. 5, the description is made on the assumption that the reproduction of a video by the user (user 1) has never been performed. As shown in FIG. 5, a user field 71 and an image list display field 72 are provided in the reproduction image selection screen 69. In addition, a cursor 73 which is moved in the screen according to the operation of the operation unit 15 is displayed in the reproduction image selection screen 69.

In the user field 71, a user name (user ID is also possible) of the user using the medical image processing device 14 is displayed. The reproduction control unit 44A causes the user name to be displayed in the user field 71 on the basis of the user specifying result input from the user specifying unit 42.

In the image list display field 72, a thumbnail image 38S of the video 38 stored in each folder in the image storage unit 53, a video reproduction button 75, and a reproduction number display 77 which will be described below are displayed. In a case where the reproduction image selection screen 69 is displayed, the reproduction control unit 44A generates the thumbnail image 38S of each video 38 in the image storage unit 53. For example, in a case where the static image 39 is stored in the same folder as the video 38, the reproduction control unit 44A generates the thumbnail image 38S on the basis of the static image 39, and conversely, in a case where the static image 39 is not stored, the reproduction control unit 44A generates the thumbnail image 38S on the basis of any frame image 38a of the video 38.

Next, the reproduction control unit 44A causes the thumbnail image 38S of each video 38 to be displayed in the image list display field 72. In a case where the thumbnail images 38S of all of the videos 38 cannot be displayed in the image list display field 72, the reproduction control unit 44A switches the thumbnail images 38S to be displayed in the image list display field 72 whenever a selection operation of selecting a display switch button 72a of the image list display field 72 is performed by the cursor 73 via the operation unit 15.

The reproduction control unit 44A causes a folder name of the video 38 (file name of the video 38 is also possible) corresponding to each thumbnail image 38S, and the video reproduction button 75 to be displayed for each thumbnail image 38S in the image list display field 72.

The information display control unit 44B causes the reproduction number display 77 indicating the reproduction number of each video 38 by the user to be displayed for each thumbnail image 38S in the image list display field 72 by referring to the reproduction number storage unit 54, which will be described below, in the storage unit 47. In this manner, in a case where a plurality of thumbnail images 38S are present in the image list display field 72, it is possible to display the reproduction number display 77 of the corresponding video 38 for each thumbnail image 38S in an association manner.

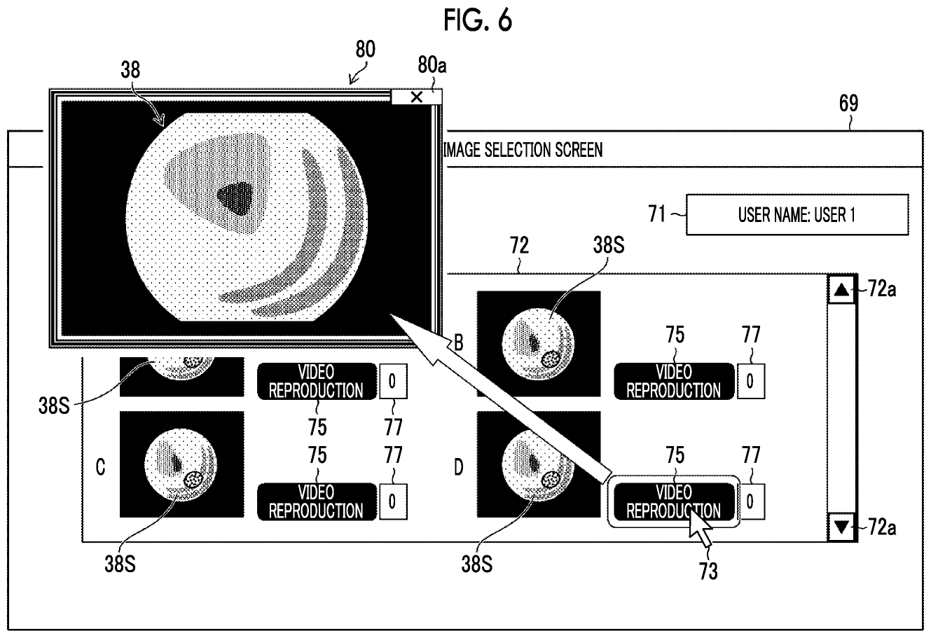

FIG. 6 is an explanatory diagram for describing the reproduction control of the video 38 by the reproduction control unit 44A. As shown in FIG. 6, in a case where a selection operation of selecting any video reproduction button 75 in the image list display field 72 is performed by the cursor 73 via the operation unit 15, the reproduction control unit 44A acquires the video 38 corresponding to the selection operation from the image storage unit 53, and causes the display device 16 to execute video reproduction based on the video 38. In this manner, in this embodiment, the display device 16 displays a video reproduction screen 80 in a window different from the reproduction image selection screen 69, and the reproduction of the video 38 is started in the video reproduction screen 80. In a case where the capturing of the static image 39 is performed, the video 38 for one minute before the capturing of the static image 39 to one minute after the capturing of the static image 39 may be reproduced.

Here, the reproduction aspect of the video 38 is not particularly limited, and the display device 16 may perform full screen display or the like of the video reproduction screen 80, for example.

In a case where a closing operation of selecting a closing button 80a in the video reproduction screen 80 is performed by the cursor 73 via the operation unit 15, the reproduction control unit 44A closes the video reproduction screen 80. In this manner, it is possible to end the reproduction of the video 38 at any timing such as during the reproduction of the video 38 or after the full video 38 is reproduced.

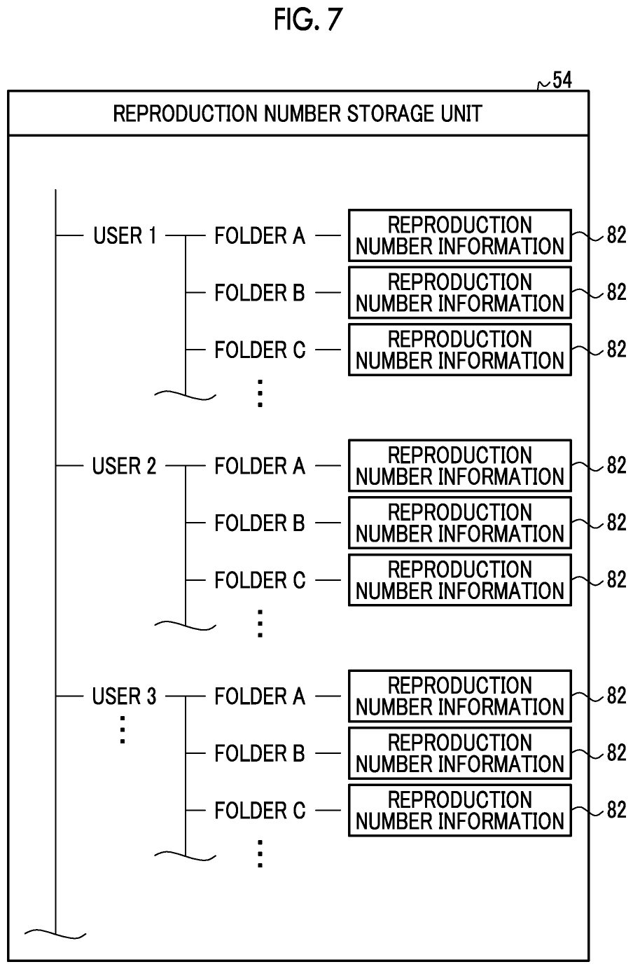

FIG. 7 is a schematic diagram of the reproduction number storage unit 54 in the storage unit 47. As shown in FIG. 7, the reproduction number storage unit 54 is used in the counting of the reproduction number of the video 38 by the reproduction number control unit 45, which will be described below, and the storage of the counting results of the reproduction number. The reproduction number storage unit 54 individually stores reproduction number information 82 indicating the reproduction number of each video 38 in each folder in the image storage unit 53, for each user (user 1, user 2, user 3, and the like). In this manner, it is possible to discriminate the reproduction number of each video 38 for each user by referring to the reproduction number storage unit 54. Then, each piece of the reproduction number information 82 is updated by the reproduction number control unit 45 which will be described below.

Returning to FIG. 2, in a case where the reproduction of the video 38 is started by the reproduction control unit 44A, the reproduction number control unit 45 is operated to individually count the reproduction number of each video 38 for each user.

First, the reproduction number control unit 45 acquires the reproduction number of the video 38 by the user, of which the reproduction is started by the reproduction control unit 44A, by referring to the reproduction number storage unit 54, on the basis of the user specifying result input from the user specifying unit 42 and the type of the video 38 (folder name). Then, in a case where the video 38 is reproduced until the specific frame image 60 among a plurality of frame images 38a constituting the video 38 of which the reproduction is started, the reproduction number control unit 45 counts up the reproduction number of the video 38 on the basis of the specific frame information 56 in the image storage unit 53 which corresponds to the video 38.

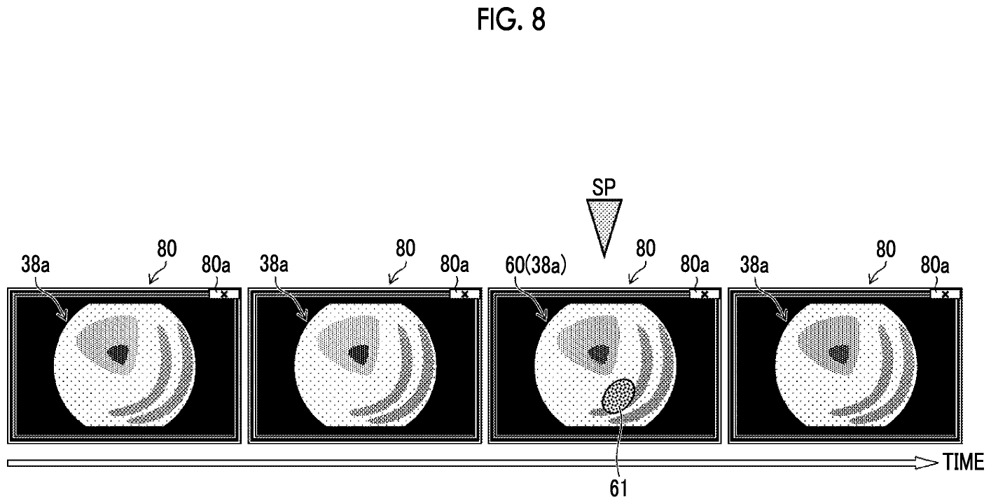

FIG. 8 is an explanatory diagram for describing counting-up of the reproduction number of the video 38 by the reproduction number control unit 45 in a case where the specific frame image 60 is specified by the medical image analysis processing unit 43A. It is assumed that the reproduction time of the video 38 elapses from the left side to the right side in the drawing.

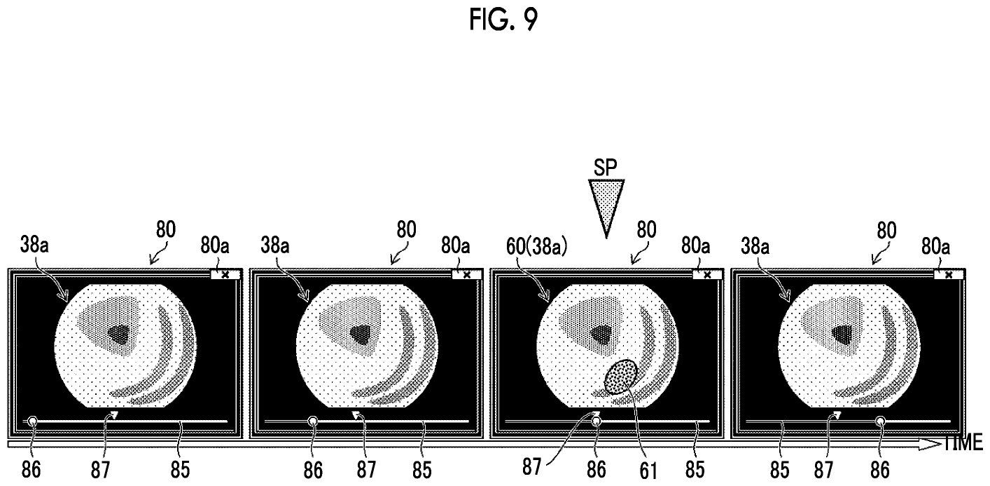

FIG. 9 is an explanatory diagram for describing counting-up of the reproduction number of the video 38 by the reproduction number control unit 45 in a case where the specific frame image 60 is specified by the image specifying unit 43B. In this case, it is preferable that the reproduction control unit 44A displays a seekbar 85, a movement index 86, and a fixed index 87 in the video reproduction screen 80.

The seekbar 85 and the movement index 86 indicate which part of the entire reproduction time (frame image 38a) of the video 38 is being reproduced. The fixed index 87 is an index indicating the reproduction timing of the specific frame image 60 in the entire reproduction time (frame image 38a) of the video 38. The position of the fixed index 87 on the seekbar 85 is determined on the basis of the specific frame information 56 corresponding to the video 38. The same display may be performed in the video reproduction screen 80 shown in FIG. 8.

As shown in FIGS. 8 and 9, in a case where the reproduction of the video 38 is started by the reproduction control unit 44A, the reproduction number control unit 45 does not count up the reproduction number of the video 38 until the specific frame image 60 is reproduced among the frame images 38a constituting the video 38, on the basis of the specific frame information 56 corresponding to the video 38. Then, the reproduction number control unit 45 counts up (+1) the reproduction number of the video 38 in a case where the reproduction of the video 38 is continued until the specific frame image 60 as indicated by an arrow SP in the drawing. That is, in a case where the video 38 is reproduced until the specific frame image 60, the reproduction number of the video 38 is counted up even if the reproduction of the video is ended at any timing after reproducing the specific frame image 60.

Here, in a case where a plurality of specific frame images 60 are included in one video 38, when the last specific frame image 60 is reproduced, that is, when all of the specific frame images 60 are reproduced, the reproduction number control unit 45 counts up the reproduction number of the video 38. In a case where the first specific frame image 60 is reproduced, or in a case where the specific frame images 60 as a certain ratio or more of all of the specific frame images 60 are reproduced, the reproduction number of the video 38 may be counted up. Further, in a case where all of the specific frame images 60 specified by predetermined one of the medical image analysis processing unit 43A and the image specifying unit 43B are reproduced, the reproduction number of the video 38 may be counted up.

In a case where the reproduction of the video 38 is ended and the reproduction number of the video 38 is counted up, the reproduction number control unit 45 stores the reproduction number after the counting-up in the reproduction number information 82 in the reproduction number storage unit 54 which corresponds to both the user and the type of the video 38. In this manner, in the reproduction number storage unit 54, the reproduction number information 82 is individually stored and updated for each user and for each type of the video 38.

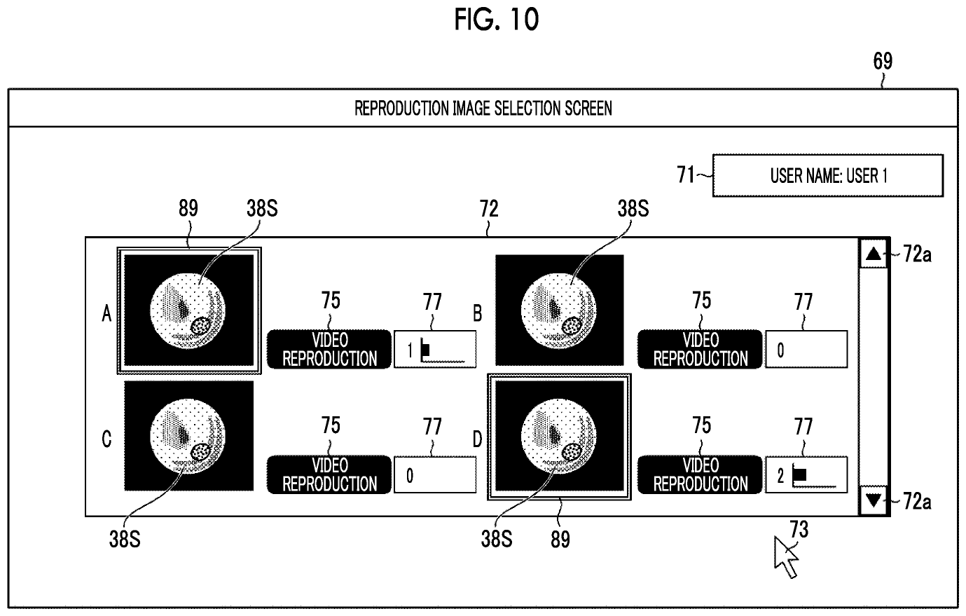

FIG. 10 is an explanatory diagram for describing updating of the reproduction number display 77 by the information display control unit 44B. As shown in FIG. 10, in a case where the reproduction number information 82 is updated by the reproduction number control unit 45, the information display control unit 44B updates the reproduction number display 77 in the image list display field 72 on the basis of the updated reproduction number information 82.

The information display control unit 44B displays the reproduction number display 77 corresponding to the video 38 (thumbnail image 38S) of which the reproduction number is “0” with a “numerical value”, and displays the reproduction number display 77 corresponding to the video 38 (thumbnail image 38S) of which the reproduction number is “1” or more with both a “numerical value” and a “graph”. The reproduction number display 77 may be displayed with at least one of the “numerical value” or the “graph” regardless of the reproduction number of the video 38.

For the thumbnail image 38S of which the reproduction number is “1” or more, the information display control unit 44B performs frame display 89 for surrounding the thumbnail image 38S in the image list display field 72. The presence or absence of the frame display 89 indicates the presence or absence of the reproduction of the video 38 corresponding to the thumbnail image 38S displayed in the image list display field 72. Therefore, the frame display 89 corresponds to reproduction presence/absence information of an embodiment of the invention. The presence or absence of the reproduction of the video 38 may be discriminated by other display aspects or display methods other than the frame display 89.

[Operation of Endoscope System]

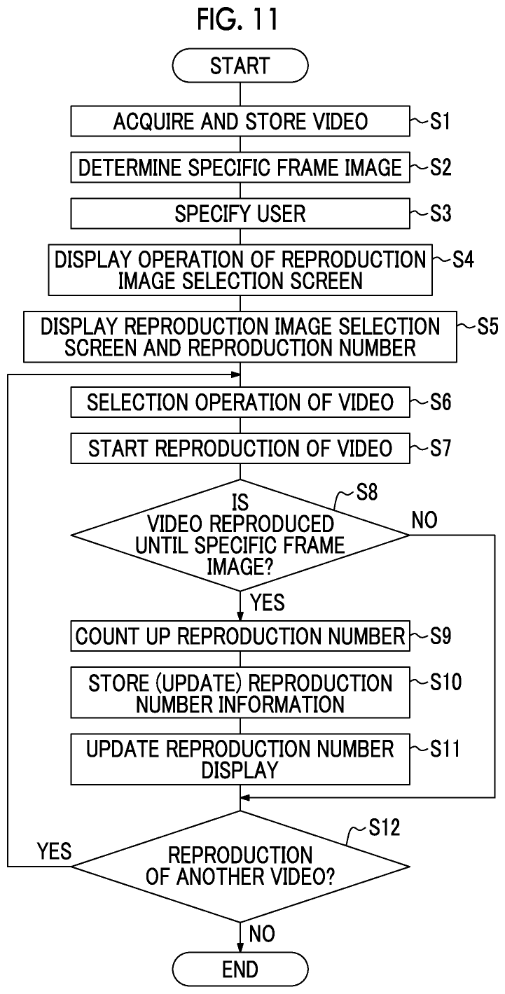

FIG. 11 is a flowchart showing a flow of reproducing the video 38 and counting the reproduction number of the video 38 (medical image processing method) in the endoscope system 9. As shown in FIG. 11, prior to the reproduction of the video 38 by the medical image processing device 14, the image acquisition unit 41 acquires the video 38 from the processor 12 or the like, and individually stores the acquired video 38 for each folder in the image storage unit 53 (step S1).

Whenever a new video 38 is stored in the image storage unit 53 by the image acquisition unit 41, the medical image analysis processing unit 43A and the image specifying unit 43B are operated. By the image analysis process using deep learning described above, the medical image analysis processing unit 43A analyzes whether the region of interest 61 is included for each frame image 38a of the video 38 and determines the frame image 38a including the region of interest 61 as the specific frame image 60 (step S2). In this manner, it is possible to determine the frame image 38a including the region of interest 61, which is highly necessary to be checked for an accurate diagnosis by the user (doctor), as the specific frame image 60.

Meanwhile, the image specifying unit 43B checks whether the static image 39 is stored in the same folder as the newly stored video 38. Then, in a case where the static image 39 is stored in the same folder as the video 38, the image specifying unit 43B determines, as the specific frame image 60, the frame image 38a captured at the imaging timing at which the static image 39 is captured, on the basis of the header information 39a of the static image 39 (step S2). In this manner, it is possible to determine the frame image 38a having a high possibility of the region of interest 61 being included, as the specific frame image 60.

The medical image analysis processing unit 43A and the image specifying unit 43B store the specific frame information 56 relating to the determined specific frame image 60, in the folder corresponding to the new video 38 in the image storage unit 53.

In a case where the user performs a start operation (login operation) for using the medical image processing device 14 by the operation unit 15, the user specifying unit 42 controls the display control unit 44 to cause the display device 16 to display the login screen. In a case where, by receiving this display, the user performs the login operation of inputting the user ID and the password by the operation unit 15, the user specifying unit 42 collates the received user ID and password with the user information 50 in the storage unit 47 to specify the user (step S3). Then, the user specifying unit 42 outputs the user specifying result to each of the display control unit 44 and the reproduction number control unit 45.

Next, in a case where the user performs the reproduction image selection screen display operation by the operation unit 15 (step S4), the reproduction control unit 44A causes the display device 16 to display the reproduction image selection screen 69 on the basis of the program 51 and the image storage unit 53 in the storage unit 47 (step S5). In this case, the information display control unit 44B displays the reproduction number display 77 of each video 38 corresponding to the user, for each thumbnail image 38S in the image list display field 72 in an association manner, by referring to the reproduction number storage unit 54 on the basis of the user specifying result by the user specifying unit 42 (step S5). In this manner, it is possible to easily grasp the reproduction status of each video 38 of the user.

In a case where the user performs the selection operation of selecting any video reproduction button 75 by the cursor 73 via the operation unit 15, the reproduction control unit 44A acquires the video 38 corresponding to the selection operation from the image storage unit 53 (step S6). Then, the reproduction control unit 44A causes the display device 16 to execute video reproduction on the video reproduction screen 80, on the basis of the video 38 acquired from the image storage unit 53 (step S7, corresponding to a video reproduction control step of an embodiment of the invention). In this manner, the reproduction of the video 38 is started.

In a case where the reproduction of the video is started by the reproduction control unit 44A, the reproduction number control unit 45 acquires the reproduction number of the video 38 by the user, of which the reproduction is started by the reproduction control unit 44A, by referring to the reproduction number storage unit 54, on the basis of the user specifying result input from the user specifying unit 42 and the type of the video 38. Then, in a case where the video 38 is reproduced until the specific frame image 60, the reproduction number control unit 45 counts up the reproduction number of the video 38 of which the reproduction is started, on the basis of the specific frame information 56 corresponding to the video 38 (YES in step S8, and step S9, corresponding to a reproduction number control step of an embodiment of the invention).

Then, the reproduction number control unit 45 stores the reproduction number of the video 38 after the counting-up, in the reproduction number information 82 in the reproduction number storage unit 54 which corresponds to both the user and the type of the video 38 (step S10). In this manner, the reproduction number information 82 in the reproduction number storage unit 54 is updated. In this embodiment, since the reproduction number of the video 38 is individually counted for each user, and the reproduction number of the video 38 is individually stored for each user, it is possible to accurately grasp the reproduction status of the video 38 for each user.

After the reproduction of the video on the video reproduction screen 80 is ended, in a case where the reproduction number information 82 is updated by the reproduction number control unit 45, the information display control unit 44B updates the reproduction number display 77 in the image list display field 72 on the basis of the updated reproduction number information 82 (step S11, corresponding to a display control step of an embodiment of the invention). In this manner, the latest reproduction number of the video 38 by the user is displayed on the display device 16.

In a case where the reproduction number of the video 38 is counted up from 0 to 1, the information display control unit 44B performs the frame display 89 for the thumbnail image 38S corresponding to the video 38. In this manner, it is possible to easily discriminate the presence or absence of the reproduction of each video 38 corresponding to each thumbnail image 38S in the image list display field 72.

Meanwhile, in a case where the video 38 is not reproduced until the specific frame image 60, the reproduction number control unit 45 does not count up the reproduction number of the video 38 (NO in step S8).

Hereinafter, in a case where the user performs the reproduction of another video 38 (Yes in step S12), processes from step S6 to step S11 are repeatedly executed.

### Effect of Embodiment

In a case where the video 38 is reproduced until the specific frame image 60 among the frame images 38a constituting the video 38, the endoscope system 9 of this embodiment counts up the reproduction number of the video 38. Therefore, in a case where checking the content of the video 38 by the user is substantially completed even if the full video 38 is not reproduced, it is possible to count up the reproduction number of the video 38. As a result, it is possible to accurately grasp the reproduction status of each video 38 by the user.

[Others]

In the above-described embodiment, the medical image analysis processing unit 43A determines whether the region of interest 61 is included for each frame image 38a by the image analysis process using deep learning, but may perform the determination using another method. For example, the frame image 38a is divided into a plurality of rectangular regions, and each divided rectangular region is set as a local region. Then, a feature quantity (for example, hue) of pixels in the local region is calculated for each local region of the frame image 38a. Next, from the local regions, a local region having specific hue is determined as the region of interest 61. The processes are repeatedly executed for all of the frame images 38a.

In this manner, the medical image analysis processing unit 43A can detect the region of interest 61 in the frame image 38a on the basis of the feature quantity of the pixels in the frame image 38a.

In the above-described embodiment, the medical image analysis processing unit 43A of the medical image processing device 14 analyzes whether the region of interest 61 is included for each frame image 38a, but an image analysis result for each frame image 38a performed by another device may be acquired. For example, the medical image analysis processing unit 43A acquires the image analysis result (specific frame information 56 or the like) from a recording device (storage device) that records the image analysis result for each frame image 38a. In this case, the medical image analysis processing unit 43A functions as the medical image analysis result acquisition unit that acquires the image analysis result.

In the above-described embodiment, the processor 12 and the medical image processing device 14 are separately provided, but the processor 12 and the medical image processing device 14 may be integrally provided. That is, the processor 12 may have a function as the medical image processing device 14.

In the above-described embodiment, the reproduction of the video 38 captured by the endoscope 10 is described, but the invention can be applied to various medical image processing devices which reproduce a video 38 captured by various medical devices that capture a video of an observation site, such as an ultrasound diagnostic apparatus, an X-ray image diagnosis system, digital mammography, a computed tomography (CT) inspection apparatus, and a magnetic resonance imaging (MRI) inspection apparatus. Further, the invention can be applied to various video reproduction devices that reproduce a video 38 such as an image diagnosis (analysis) device used in analysis and diagnosis of the video 38 as a medical image, and a medical service support device used in creating a medical report or the like.

[Additional Remark]

The present specification includes disclosure of various technical ideas including the inventions described below.

[Additional Remark A1]

A medical image processing device according to an embodiment of the invention further comprising: a medical image analysis processing unit that detects a region of interest, which is a region to be noticed, on the basis of a feature quantity of pixels of a medical image, and a medical image analysis result acquisition unit that acquires an analysis result of the medical image analysis processing unit.

[Additional Remark A2]

A medical image processing device according to an embodiment of the invention further comprising: a medical image analysis processing unit that detects presence or absence of a target to be noticed, on the basis of a feature quantity of pixels of a medical image, and a medical image analysis result acquisition unit that acquires an analysis result of the medical image analysis processing unit.

[Additional Remark A3]

The medical image processing device according to Additional remark A1 or A2, wherein the medical image analysis result acquisition unit acquires the analysis result from a recording device recording an analysis result of the medical image, and the analysis result includes any one or both of the region of interest that is the region to be noticed included in the medical image and presence or absence of the object to be noticed.

[Additional Remark B1]

The medical image processing device described in any one of Additional remarks A1 to A3, wherein the medical image is a normal light image that is obtained from the application of light in a white-light wavelength range or light in a plurality of wavelength ranges as the light in a white-light wavelength range.

[Additional Remark B2]

The medical image processing device according to Additional remark B1, wherein the medical image is an image that is obtained from the application of light in a specific wavelength range, and the specific wavelength range is a range narrower than the white-light wavelength range.

[Additional Remark B3]

The medical image processing device according to Additional remark B2, wherein the specific wavelength range is a blue-light wavelength range or a green-light wavelength range of a visible-light wavelength range.

[Additional Remark B4]

The medical image processing device according to Additional remark B3, wherein the specific wavelength range includes a wavelength range of 390 nm to 450 nm or 530 nm to 550 nm, and light in the specific wavelength range has a peak wavelength in a wavelength range of 390 nm to 450 nm or 530 nm to 550 nm.

[Additional Remark B5]

The medical image processing device according to Additional remark B2, wherein the specific wavelength range is a red-light wavelength range of a visible-light wavelength range.

[Additional Remark B6]

The medical image processing device according to Additional remark B5, wherein the specific wavelength range includes a wavelength range of 585 nm to 615 nm or 610 nm to 730 nm, and light in the specific wavelength range has a peak wavelength in a wavelength range of 585 nm to 615 nm or 610 nm to 730 nm.

[Additional Remark B7]

The medical image processing device according to Additional remark B2, wherein the specific wavelength range includes a wavelength range where a light absorption coefficient in oxyhemoglobin is different from that in reduced hemoglobin, and light in the specific wavelength range has a peak wavelength in a wavelength range where a light absorption coefficient in oxyhemoglobin is different from that in reduced hemoglobin.

[Additional Remark B8]

The medical image processing device according to Additional remark B7, wherein the specific wavelength range includes a wavelength range of 400±10 nm, 440±10 nm, 470±10 nm, or 600 nm to 750 nm, and light in the specific wavelength range has a peak wavelength in a wavelength range of 400±10 nm, 440±10 nm, 470±10 nm, or 600 nm to 750 nm.

[Additional Remark B9]

The medical image processing device according to Additional remark B2, wherein the medical image is an in-vivo image of the inside of a living body, and the in-vivo image has information of fluorescence emitted by fluorescent materials.

[Additional Remark B10]

The medical image processing device according to Additional remark B9, wherein the fluorescence is obtained from the application of excitation light, which has a peak wavelength in a wavelength range of 390 nm to 470 nm, to the inside of the living body.

[Additional Remark B11]

The medical image processing device according to Additional remark B2, wherein the medical image is an in-vivo image of the inside of a living body, and the specific wavelength range is an infrared wavelength range.

[Additional Remark B12]

The medical image processing device according to Additional remark B11, wherein the specific wavelength range includes a wavelength range of 790 nm to 820 nm or 905 nm to 970 nm, and light in the specific wavelength range has a peak wavelength in a wavelength range of 790 nm to 820 nm or 905 nm to 970 nm.

[Additional Remark B13]

The medical image processing device described in any one of Additional remarks A1 to A3, wherein a medical image acquisition unit comprises a special light image acquisition unit that acquires a special light image including information about the specific wavelength range on the basis of a normal light image obtained from the application of light in a white-light wavelength range or light in a plurality of wavelength ranges as the light in a white-light wavelength range, and the medical image is the special light image.

[Additional Remark B14]

The medical image processing device according to Additional remark B13, wherein a signal in the specific wavelength range is obtained from an arithmetic operation based on color information about red, green, and blue or cyan, magenta, and yellow included in the normal light image.

[Additional Remark B15]

The medical image processing device described in any one of Additional remarks A1 to A3 further comprising: a feature-quantity-image generation unit generating a feature quantity image from an arithmetic operation based on at least one of the normal light image that is obtained from the application of light in a white-light wavelength range or light in a plurality of wavelength ranges as the light in a white-light wavelength range and the special light image that is obtained from the application of light in a specific wavelength range, the medical image is the feature quantity image.

[Additional Remark C1]

An endoscope apparatus comprising: the medical image processing device described in any one of Additional remarks described above; and an endoscope that acquires an image from the application of at least one of light in a white-light wavelength range or light in the specific wavelength range.

[Additional Remark C2]

A diagnosis support apparatus comprising: the medical image processing device according to any one of Additional remarks described above.

[Additional Remark C3]

A medical service support apparatus comprising: the medical image processing device according to any one of Additional remarks described above.

### EXPLANATION OF REFERENCES

9: endoscope system
10: endoscope
11: light source device
12: processor
13: display device
14: medical image processing device
16: display device
20: insertion part
21: operation part
22: universal cord
25: soft portion
26: bendable portion
27: distal end portion
28: image pick-up element
29: bendable operation knob
30: air/water supply button
31: suction button
32: static image capturing instruction portion
33: treatment tool inlet
35: light guide
36: signal cable
37A: connector
37B: connector
38: video
38S: thumbnail image
38a: frame image
39: static image
39a: header information
41: image acquisition unit
42: user specifying unit
43A: medical image analysis processing unit
43B: image specifying unit
44: display control unit
44A: reproduction control unit
44B: information display control unit
45: reproduction number control unit
47: storage unit
50: user information
51: program
53: image storage unit
54: reproduction number storage unit
56: specific frame information
60: specific frame image
61: region of interest
65: deep learning algorithm
69: reproduction image selection screen
71: user field
72: image list display field
72a: display switch button
73: cursor
75: video reproduction button
77: reproduction number display
80: video reproduction screen
80a: closing button
82: reproduction number information
85: seekbar
86: movement index
87: fixed index
89: frame display
S1 to S12: step (medical image processing method)
SP: arrow

What is claimed is:

1. A medical image processing device comprising:
a video reproduction control unit that controls reproduction of a video of a medical image in a display device;
a reproduction number control unit that counts the reproduction number of the video by the video reproduction control unit, and counts up the reproduction number of the video in a case where the video is reproduced until a specific frame image among a plurality of frame images constituting the video; and
an information display control unit that causes the display device to display the reproduction number of the video, which is counted by the reproduction number control unit.

2. The medical image processing device according to claim 1, further comprising:
a medical image analysis processing unit that analyzes whether a region of interest as a region to be noticed is included for each of the frame images, and determines the frame image including the region of interest as the specific frame image.

3. The medical image processing device according to claim 2,
wherein the medical image analysis processing unit analyzes whether the region of interest is included in the frame image by an image analysis process using deep learning.

4. The medical image processing device according to claim 1,
wherein the video is acquired by a medical device having a function of performing capturing of the video and capturing of a static image during the capturing of the video, and
the medical image processing device further comprises
a timing information storage unit that stores timing information indicating a timing at which the capturing of the static image is executed during the capturing of the video in a case where the capturing of the static image is executed by the medical device, and
an image specifying unit that determines the frame image captured at the timing at which the capturing of the static image is executed, as the specific frame image on the basis of the timing information stored in the timing information storage unit.

5. The medical image processing device according to claim 1, further comprising:
a reproduction number storage unit that stores the reproduction number of the video, which is counted by the reproduction number control unit.

6. The medical image processing device according to claim 2, further comprising:
a reproduction number storage unit that stores the reproduction number of the video, which is counted by the reproduction number control unit.

7. The medical image processing device according to claim 3, further comprising:
a reproduction number storage unit that stores the reproduction number of the video, which is counted by the reproduction number control unit.

8. The medical image processing device according to claim 4, further comprising:
a reproduction number storage unit that stores the reproduction number of the video, which is counted by the reproduction number control unit.

9. The medical image processing device according to claim 5, further comprising:
a user specifying unit that specifies a user who executes reproduction of the video in the video reproduction control unit,
wherein the reproduction number control unit individually counts the reproduction number of the video for each user, and
the reproduction number storage unit individually stores the reproduction number of the video for each user.

10. The medical image processing device according to claim 6, further comprising:
a user specifying unit that specifies a user who executes reproduction of the video in the video reproduction control unit,
wherein the reproduction number control unit individually counts the reproduction number of the video for each user, and
the reproduction number storage unit individually stores the reproduction number of the video for each user.

11. The medical image processing device according to claim 7, further comprising:
a user specifying unit that specifies a user who executes reproduction of the video in the video reproduction control unit,
wherein the reproduction number control unit individually counts the reproduction number of the video for each user, and
the reproduction number storage unit individually stores the reproduction number of the video for each user.

12. The medical image processing device according to claim 8, further comprising:
a user specifying unit that specifies a user who executes reproduction of the video in the video reproduction control unit,
wherein the reproduction number control unit individually counts the reproduction number of the video for each user, and
the reproduction number storage unit individually stores the reproduction number of the video for each user.

13. The medical image processing device according to claim 1,
wherein the information display control unit causes the display device to display reproduction presence/absence information indicating presence or absence of reproduction of the video.

14. The medical image processing device according to claim 2,
wherein the information display control unit causes the display device to display reproduction presence/absence information indicating presence or absence of reproduction of the video.

15. The medical image processing device according to claim 3,
wherein the information display control unit causes the display device to display reproduction presence/absence information indicating presence or absence of reproduction of the video.

16. The medical image processing device according to claim 4,
wherein the information display control unit causes the display device to display reproduction presence/absence information indicating presence or absence of reproduction of the video.

17. The medical image processing device according to claim 5,
wherein the information display control unit causes the display device to display reproduction presence/absence information indicating presence or absence of reproduction of the video.

18. The medical image processing device according to claim 1,
wherein the information display control unit causes the display device to display at least one of a numerical value or a graph indicating the reproduction number of the video, as the reproduction number of the video.

19. The medical image processing device according to claim 1,
wherein in a case where a plurality of videos are present, the information display control unit causes the display device to display the reproduction number of the video, for each of the videos.

20. A medical image processing method comprising:
a video reproduction control step of controlling reproduction of a video of a medical image in a display device;
a reproduction number control step of counting the reproduction number of the video reproduced in the video reproduction control step, and counting up the reproduction number of the video in a case where the video is reproduced until a specific frame image among a plurality of frame images constituting the video; and
a display control step of causing the display device to display the reproduction number of the video, which is counted in the reproduction number control step.
